(19) United States
(12) Patent Application Publication
(10) Pub. No.: US 2009/0099630 A1
(43) Pub. Date: Apr. 16, 2009

(54) TUCKABLE ELECTRIC WARMING BLANKET FOR PATIENT WARMING

(75) Inventors: Scott D. Augustine, Bloomington, MN (US); Ryan S. Augustine, Minneapolis, MN (US); Randall C. Arnold, Minnetonka, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: AUGUSTINE BIOMEDICAL AND DESIGN LLC, Eden Prairie, MN (US)

(21) Appl. No.: 12/251,317

(22) Filed: Oct. 14, 2008

Related U.S. Application Data
(60) Provisional application No. 60/979,681, filed on Oct. 12, 2007.

(51) Int. Cl. A61F 7/08; H05B 3/02

(52) U.S. Cl. 607/96; 219/212

(57) ABSTRACT

A tuckable electric warming blanket for patient warming and a method of using such a warming blanket. The blanket may be used to warm the lower body of the patient or other portion of the patient's body. The blanket may include one or more rigid stays that extend along the right and left sides of the blanket. The stays assist in fully tucking the blanket under the patient and reduce the potential for bunching up the heating element under the patient. The blanket may also include a flexible, unheated foot portion that is tuckable about the patient's feet.

### PRIORITY CLAIM

The present application claims priority to provisional application Ser. No. 60/979,681, entitled ELECTRIC WARMING BLANKET FOR LOWER BODY PATIENT WARMING filed on Oct. 12, 2007; the specification of which is incorporated by reference in its entirety herein.

### TECHNICAL FIELD

The present invention is related to heating or warming blankets or pads and more particularly to those including electrical heating elements.

### BACKGROUND

It is well established that surgical patients under anesthesia become poikilothermic. This means that the patients lose their ability to control their body temperature and will take on or lose heat depending on the temperature of the environment. Since modern operating rooms are all air conditioned to a relatively low temperature for surgeon comfort, the majority of patients undergoing general anesthesia will lose heat and become clinically hypothermic if not warmed.

Over the past 15 years, forced-air warming (FAW) has become the “standard of care” for preventing and treating the hypothermia caused by anesthesia and surgery. FAW consists of a large heater/blower attached by a hose to an inflatable air blanket. The warm air is distributed over the patient within the chambers of the blanket and then is exhausted onto the patient through holes in the bottom surface of the blanket.

Although FAW is clinically effective, it suffers from several problems including: a relatively high price; air blowing in the operating room, which can be noisy and can potentially contaminate the surgical field; and bulkiness, which, at times, may obscure the view of the surgeon. Moreover, the low specific heat of air and the rapid loss of heat from air require that the temperature of the air, as it leaves the hose, be dangerously high—in some products as high as 45° C. This poses significant dangers for the patient. Second and third degree burns have occurred both because of contact between the hose and the patient's skin, and by blowing hot air directly from the hose onto the skin without connecting a blanket to the hose. This condition is common enough to have its own name—“hosing.” The manufacturers of forced air warming equipment actively warn their users against hosing and the risks it poses to the patient.

To overcome the aforementioned problems with FAW, several companies have developed electric warming blankets. However, these electric blankets have a number of inadequacies, for example, the risk of heat and pressure injuries that may be suffered by a patient improperly coming into contact with the electrical heating elements of these blankets. It is well established that heat and pressure applied to the skin can rapidly cause thermal injury to that skin. Such contact may arise if a patient inadvertently lies on an edge of a heated blanket, if a clinician improperly positions an anesthetized patient atop a portion of the heated blanket, or if a clinician tucks an edge of the blanket about the patient. Thus, there is a need for a heating blanket that effectively forms a cocoon about a patient, in order to provide maximum efficacy in heating, without posing the risk of burning the patient.

There is also a need for electrically heated blankets or pads that can be used to safely and effectively to warm patients undergoing surgery or other medical treatments. These blankets need to be flexible in order to effectively drape over the patient (making excellent contact for conductive heat transfer and maximizing the area of the patient's skin receiving conductive as well as radiant heat transfer), and should incorporate means for precise temperature control.

Electric blankets are used to maintain a patient's body temperature in a wide variety of surgical procedures. The sterile surgical field in each procedure can be quite different, and electric blankets of varying sizes and shapes are needed in order to cover a maximum amount of body surface area surface outside the surgical field. For example, a blanket that only covers a lower abdomen and legs of a patient can be used during upper body surgeries. Similarly, a blanket that covers outstretched arms and a chest area of a patient is useful for patients undergoing lower body surgery. The heat of an electric blanket can be contained by tucking the blanket beneath the patient. For instance, the heat of a lower body electric blanket can be contained by tucking the blanket beneath the patient's hips, legs, and feet. However, such tucking can create dangerous gathering or folding of the blanket which may be unnoticed and undetected at this unseen location beneath the patient. Such folds could lead to overheating of the blanket and burning of the patient. If the blanket remains untucked, airflow under the blanket may lead to undesirable heat loss.

Accordingly, there remains a need for an electric heating blanket which can be safely and easily tucked beneath a patient. Furthermore, there remains a need for heating blanket which helps prevent folding of the heated portion of the blanket, particularly when the blanket is tucked beneath the patient.

### SUMMARY

Certain embodiments of the invention include a tuckable electric heating blanket that includes a heating element, a flexible shell covering the heating element, one or more stays, and unheated fold zones. The stays extend along right and left edges of the heating element and are relatively stiff. The fold zones are positioned between the heating element and the stays.

Some embodiments of the invention focus on an electric heating blanket shaped to cover the lower body of a patient. The blanket includes an electric heating assembly for covering the legs and hips of the patient. The blanket also includes a flexible shell having top and bottom sheets that are coupled together around the perimeter of the heating element assembly and about the outer edge of the blanket. The flexible shell also forms pockets about zones where the top and bottom sheets remain uncoupled together. The blanket also includes an unheated foot portion pocket located longitudinally of one of the longitudinal ends of the heating element assembly. The foot portion pocket providing an unheated flexible portion of the blanket tuckable about the patient's feet to retain heat under the blanket.

Embodiments of the invention also include a method of using a tuckable electric heating blanket. The method includes placing a patient on a surface with the patient's body extended against the surface. The method also includes placing the electric heating blanket over the patient's body where the blanket includes a flexible heating element and longitudinal stays extending along right and left edges of the heating element. The blanket also has unheated fold zones positioned between the heating element and the stays. The method includes positioning the blanket with the heating element over at least a portion of the patient's body and positioning the stays on the surface beside the portion of the patient's body. The method also includes tucking the blanket beneath the patient's body by sliding the stays over the surface and beneath the patient's body until there is resistance to advancing the stays.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1A is a schematic top view of a tuckable lower body heating blanket, according to some embodiments of the present invention;

FIG. 1B is a schematic top view of a tuckable electric heating blanket, according to some alternate embodiments of the present invention;

FIG. 2A is a side view of a portion of a lower body heating blanket draped over a lower body portion of a patient;

FIG. 2B is a side view of a portion of a lower body heating blanket draped over a lower body portion of a patient;

FIG. 2C is a side view of a portion of a lower body heating blanket draped over a lower body portion of a patient;

FIG. 2D is a side view of a portion of a lower body heating blanket draped over a lower body portion of a patient;

FIG. 3A is a plan view of a flexible heating blanket subassembly for a heating blanket, according to some embodiments of the present invention;

FIG. 3B is an end view of some embodiments of the subassembly shown in FIG. 3A;

FIG. 4A is a top plan view of a heating element assembly, according to some embodiments of the present invention, which may be incorporated in the blanket shown in FIG. 1A or 1B;

FIG. 4B is a section view through section line A-A of FIG. 4A;

FIG. 5A is a top plan view of a heating element assembly, which may be incorporated in the blanket shown in FIG. 1A or 1B; and

FIG. 5B is a cross-section view through section line 5B-5B of FIG. 5A.

### DETAILED DESCRIPTION

The following discussion is presented to enable a person skilled in the art to make and use the invention. Various modifications to the illustrated embodiments will be readily apparent to those skilled in the art, and the generic principles herein may be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the invention. Skilled artisans will recognize the examples provided herein have many useful alternatives which fall within the scope of the invention. Examples of constructions, materials, dimensions, and manufacturing processes are provided for selected elements, and all other elements employ that which is known to those of skill in the field of the invention. Those skilled in the art will recognize that many of the examples provided have suitable alternatives that can be utilized. The term ‘blanket’, used to describe embodiments of the present invention, may be considered to encompass heating blankets and pads.

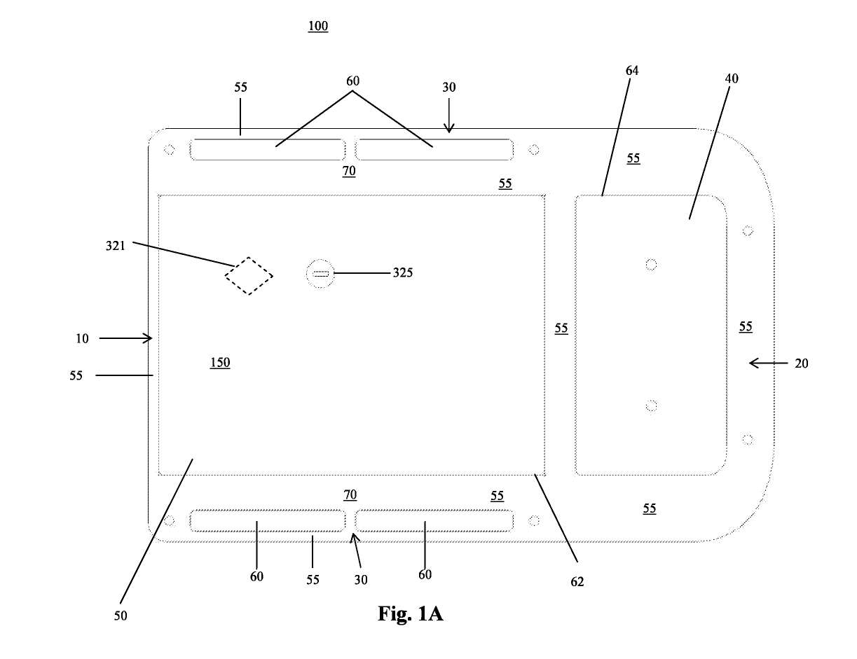

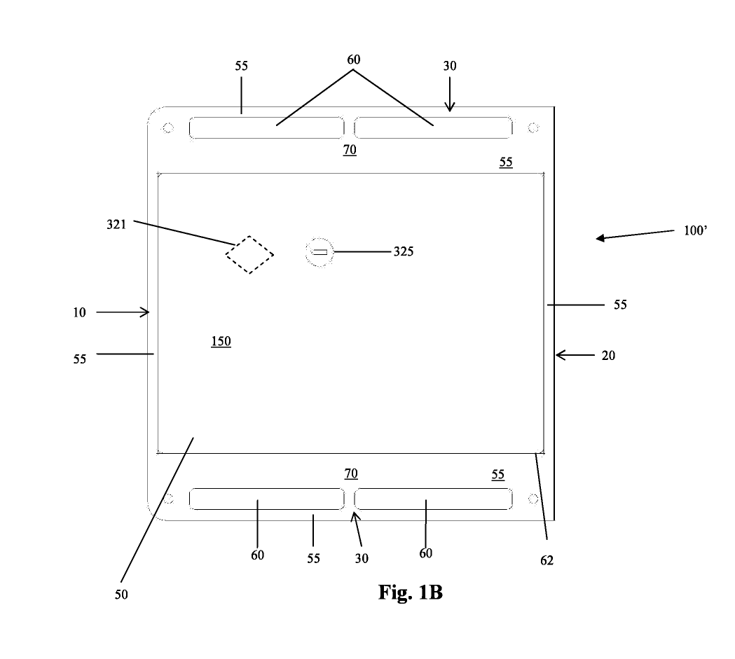

FIG. 1A is a top plan view of a tuckable lower body heating blanket 100, according to some embodiments of the present invention, which may be used to keep a patient warm during surgery. FIG. 1A illustrates blanket 100 which is approximately rectangular in shape and is generally sized to cover the lower body of a patient including the hips, legs, and feet. Blanket 100 has a proximal end 10, a distal end 20 and two opposing side edges 30. A portion of the blanket 100 is heated and includes a heating element assembly 150 which generally covers the patient's hips and legs. Heating element assembly 150 is generally rectangular and is outlined by rectangle 62. Distal to the heating element assembly 150 is a foot portion 40 which is unheated and generally covers the patient's feet. Also present on the top surface of blanket 100 is an electrical connector housing 325, described below. The general location of a temperature sensor assembly 321 is indicated generally in FIG. 1A. Such a temperature sensor assembly, described further below, is located interior to the blanket 100.

The heating element assembly 150 is covered by a flexible shell 50. Shell 50 protects and isolates the heating element assembly 150 from an external environment of blanket 100 and may further protect a patient disposed beneath blanket 100 from electrical shock hazards. According to preferred embodiments of the present invention, shell 50 is waterproof to prevent fluids, for example, bodily fluids, IV fluids, or cleaning fluids, from contacting assembly 150 and may further include an anti-microbial element, for example, being a SILVERion™ antimicrobial fabric available from Domestic Fabrics Corporation, or Ultra-Fresh™ from Thomson Research Associates.

According to some embodiments of the present invention, shell 50 includes top and bottom sheets extending over either side of assembly 150; the two sheets of shell 50 are coupled together along a seal zone 55 that extends around the perimeter of heating element assembly 150 as well as about the outer edge of blanket 100. Between the perimeter of the heating element assembly and the outer edges of the blanket 100, the shell forms various zones, or pockets, where gaps exist between the two sheets. Seal zone 55 creates a perimeter seal for the shell 50 instead of laminating the entire interior surfaces of the shell 50 to assembly 150. According to an exemplary embodiment of the present invention, shell 50 comprises PVC film. In an alternate embodiment, shell 50 comprises a nylon fabric having an overlay of polyurethane coating to provide waterproofing; the coating is on at least an inner surface of each of the two sheets, further facilitating a heat seal between the two sheets, for example, along the seal zone, according to preferred embodiments. It should be noted that, according to alternate embodiments of the present invention, a covering for heating assemblies, such as heating assembly 150, may be removable and, thus, include a reversible closure facilitating removal of a heating assembly therefrom and insertion of the same or another heating assembly therein.

FIG. 1A illustrates shell 50 forming pockets lateral to the heating element assembly 150 and adjacent to the side edges 30 of blanket 100. Within these pockets is a stiff material such as a thin plastic sheet or flexible PVC film (e.g., 0.080 inches thick) of an appropriate durometer such that the pockets function as rigid or inflexible stays 60. The stays 60 prevent inadvertent rucking of the blanket 100, that is, the folding of the heated portion (heating element 310 described below) of blanket 100 over on itself which could lead to overheating of a portion of blanket 100. In the embodiment shown in FIG. 1, there are two stays 60 extending longitudinally within each side edge 30 of blanket 100. A single stay 60 or more than two stays 60 could also be used in each side edge 30. However, the use of a single stay 60 would prevent the blanket 100 from being folded back upon itself throughout the length of the stay 60. In contrast, by using more than one stay 60, the gap between the stays 60 allows for the blanket to be folded back upon itself, such as to allow unobstructed access to the patient's lower body during surgery and to facilitate folding for storage, packaging, and transport. The elongated area between the heating element assembly 150 and the stays 60, the seal zone 55 forms a fold zone 70 which is used for tucking the side edges 30 of blanket 100 beneath the patient as described below.

FIG. 1B is a schematic top view of a tuckable electric heating blanket 100′, according to some alternate embodiments of the present invention. Elements of blanket 100′ similar to those in blanket 100 are numbered identically and need not be described separately. In contrast to blanket 100, blanket 100′ does not have a foot portion 40. That is, distal to the heating element assembly 150 is the distal end 20 of blanket 100′. All description herein of blanket 100 applies equally to blanket 100′, except for the presence or use of foot portion 40. Without foot portion 40, blanket 100′ is more easily placed in any location on the patient's body than is blanket 100. However, it is understood that blanket 100 may also be placed on positions other than the lower body of the patient.

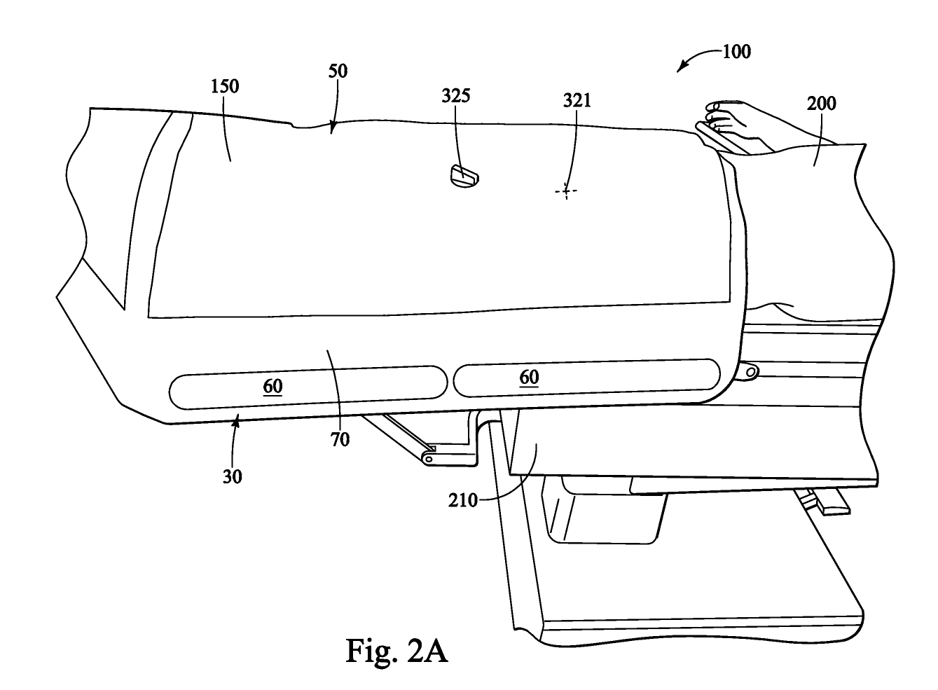

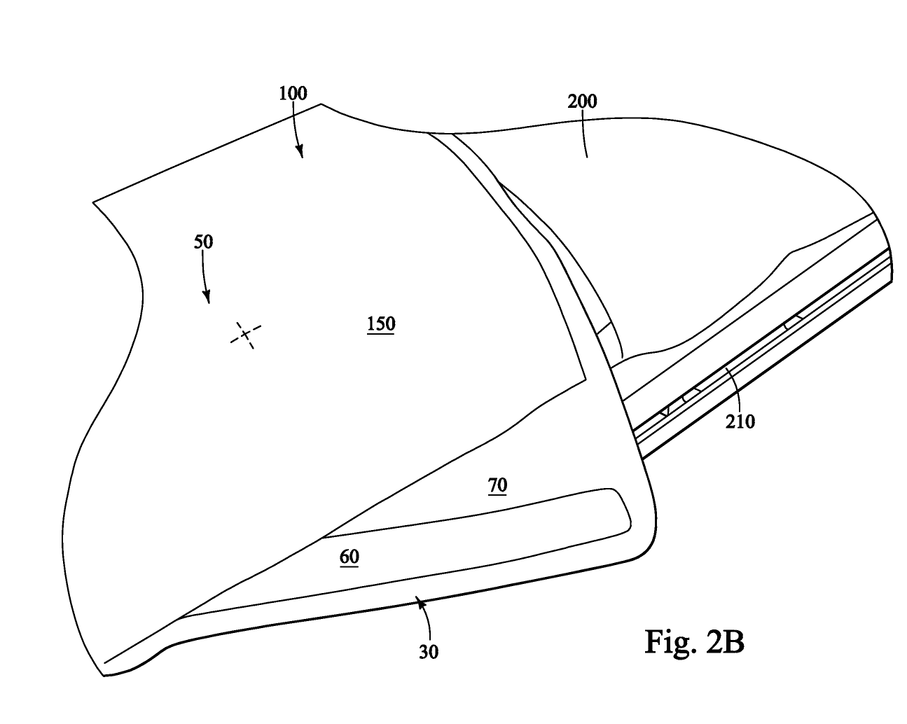

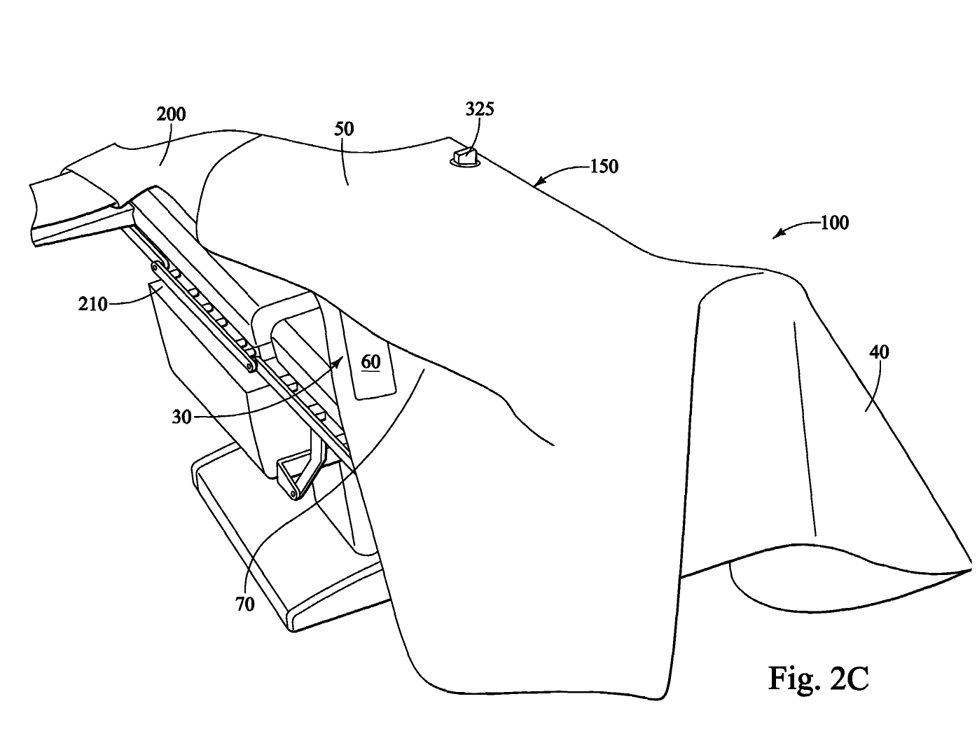

FIG. 2A shows blanket 100 lying atop the lower body portion of a patient 200 while the patient 200 is on an operating table 210. The electrical connector housing 325 and the position of temperature sensor assembly 321 are seen in phantom on the upper surface of the blanket 100. The blanket 100 drapes across and completely covers the patient's lower body, including the sides of the patient's lower body. The blanket 100 is sufficiently wide that the side edges 30 of the blanket extend around and beyond the sides of the patient's lower body to hang adjacent to the sides of the table 210. Fold zone 70 and stays 60 are shown on the side edge 30 of blanket 100.

FIGS. 2B and 2C demonstrate the side edge 30 of the blanket 100 partially tucked beneath the patient 200, between the patient 200 and the top surface of the table 210. The heating element assembly 150 within the shell 50 of the blanket 100 extends across the patients lower body and around the patient's sides to provide warmth to the patient 200. Stays 60 provide stiffness to the side edge 30 of blanket, making it easier to slide the stays over the surface of the table to tuck the side edge 30 of blanket 100 beneath the patient's lower body. That is, the stiffness of the stays 60 on the side edge 30 of blanket 100 facilitate easier tucking by increasing the amount of blanket 100 tucked in each tucking motion, thereby reducing the number of tucking motions required to tuck the entire side edge 30 of the blanket 100.

As the blanket 100 is tucked around the patient 200, the blanket 100 folds back upon itself in the fold zone 70. Because the stays 60 are stiff, they do not fold back upon themselves and thus prevent the blanket 100 from being tucked further beneath the sides of the patient 200. In this way, the stays 60 determine the maximum distance of tucking the blanket 100 and act as a stopping point by providing resistance to further advancing the stays 60.

By tucking the blanket 100 under the patient 200 completely until hitting this resistance or stopping point, the portion of the blanket 100 which includes the heating element assembly 150 is less likely to be tucked under the patient or to be folded back upon itself. Such folds are potentially dangerous and could potentially cause patient burns in some designs. Furthermore, because such folds would occur when the blanket 100 is tucked beneath the patient 200, where the tucked portion of the blanket 100 cannot be seen, such folds are particularly difficult to detect and therefore to prevent, making the stays 60 particularly useful for enhancing patient safety. Without the stopping point provided by the stays 60 in combination with the fold zone 70, the person tucking the blanket 100 would be uncertain how far it was necessary to tuck the side edges 30 of the blanket 100 beneath the patient 200 and could potentially stop tucking the blanket 100 before any bunching or extra folding in the fold zone 70 (e.g., other than the one fold back on itself) are eliminated. Accordingly, the presence of the stays 60 and the fold zone 70 help set maximum and minimum tucking limits that place the blanket 100 in the proper position relative to the patient 200. In addition, the stiffness of the stays 60 in the longitudinal direction prevents bunching of the blanket upon itself. In this way, the stays 60 help prevent the formation of folds either longitudinally or widthwise.

FIG. 2C also shows the foot portion 40 of blanket 100 draped over and beyond the patient's feet and hanging adjacent to the end of the table 210. In this embodiment, the foot portion 40 of blanket 100 is unheated and is comprised of a generally rectangular pocket (outlined as rectangle 64 in FIG. 1) in shell 50 positioned distal to the heating element. By forming a pocket in the shell 50, the blanket 100 is more flexible in the foot portion than it would be if it contained a heating element assembly or a seal zone. This allows the foot zone to bend and more closely conform to the shape of the patient's feet. Enclosing the feet within the tucked blanket 100 helps retain heat under the blanket 100, thereby avoiding heat loss to the surrounding environment. The large flexible foot portion 40 allows clinicians to fully enclose the feet. Since feet are natural radiators for heat loss, the tuckable foot portion 40 helps reduce the amount of heat lost from the feet to the environment.

In FIG. 2D, the blanket 100 is tucked completely beneath the patient's legs, between the patient 200 and the top of the table 210. The side edge 30 of the blanket 100, lateral to the stays 60 (unseen beneath the patient 200) can be partially seen in FIG. 2D as the blanket folds back upon itself. The foot portion 40 extends over and around the top of the patient's feet with the distal end 20 of the blanket 100 hanging over the end of the table. Alternatively, the distal end 20 of the blanket 100 could be tucked beneath the patient's feet, at the end of the table 100.

In use, the patient 200 is laid upon a flat surface such as the top of an operating room table in either a prone or a supine position with the lower body extended flat against the surface. The blanket 100 is draped over the patient's lower body such that it drapes completely across and over the patient's feet and legs and may also cover some or all of the patient's hips. In some embodiments, a disposable cover is placed over the blanket 100 to prevent direct contact between the patient 200 and the blanket 100. The portion of the blanket 100 containing the heating element assembly 150 is positioned over the patient's legs and hips while the side stays 60 lie on the table or hang over the sides of the table, depending upon the width of the table. In some embodiments, unheated portions of the blanket 100 may be left untucked, thereby hanging down along the sides of the operating table 210 to help trap heat under the blanket. However, in other embodiments at least most of the unheated portions of the blanket 100 are tucked under the patient 200 in order to trap heat under the blanket 100 and help prevent heat loss from air flow under the blanket 100. In some situation, the blanket 100 will be used while the patient is on a gurney, in a bed, or while sitting in a chair often provided in pre-operative settings. Moreover, some surgery is conducted with the patient in a sitting position.

In such embodiments, once the blanket 100 is properly positioned, the side edges 30 of the blanket are tucked beneath the patient. This may be accomplished by sliding the stays 60 across the surface, pushing them under the patient 200, until the blanket 100 is fully tucked under the patient with the blanket folding back upon itself in the fold zone 70. This will be sensed by the person tucking the blanket as the stays 60 resist bending and the stays 60 will not slide further beneath the patient. The tucking may be performed by beginning at either the proximal or the distal end 10, 20 of the blanket 100, tucking the blanket 100 under the patient 200 until reaching resistance at the stopping point when the blanket 100 is fully tucked and then continuing up or down the length of the patient's lower body as shown in FIGS. 2B and 2C, for example. The distal end 20 of the blanket 100 may optionally be tucked beneath the patient's feet or may be allowed to extend over and beyond the patient's feet. In this way, the blanket fits closely around the patient, forming a sort of cocoon to trap heat, while being prevented from folding upon itself in the zone of the blanket which contains the heating element assembly 150.

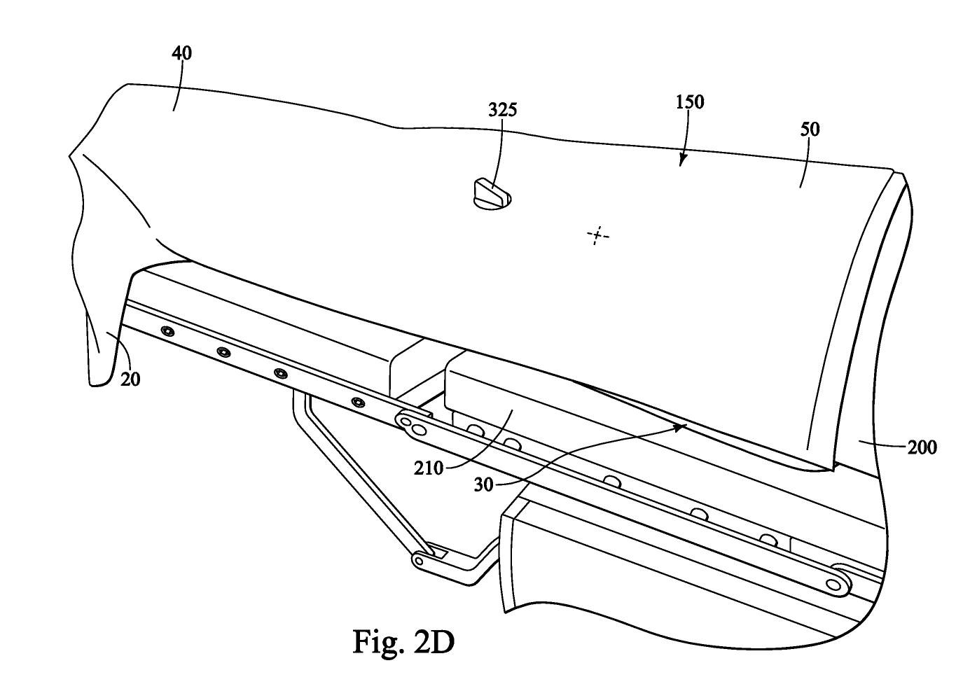

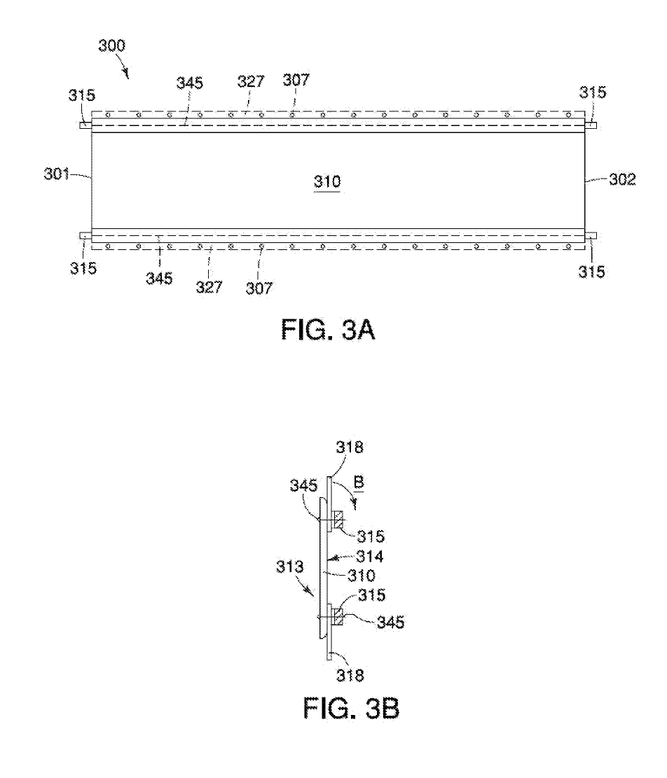

FIG. 3A is a plan view of a flexible heating blanket subassembly 300, according to some embodiments of the present invention; and FIG. 3B is an end view of some embodiments of the subassembly shown in FIG. 3A. FIG. 3A illustrates a flexible sheet-like heating element, or heater, 310 of subassembly 300 including a first end 301 and a second end 302. According to preferred embodiments of the present invention, heating element 310 comprises a conductive fabric or a fabric incorporating closely spaced conductive elements such that heating element 310 has a substantially uniform watt density output, preferably less than approximately 0.5 watts/sq. inch, and more preferably between approximately 0.2 and approximately 0.4 watts/sq. inch, across a surface area, of one or both sides 313, 314 (FIG. 3B). In some embodiments, the substantially uniform watt density output results from the generally uniform resistance per unit area that remains generally constant, independent of temperature.

Some examples of conductive fabrics which may be employed by embodiments of the present invention include, without limitation, carbon fiber fabrics, fabrics made from carbonized fibers, conductive films, or woven or non-woven non-conductive fabric or film substrates coated with a conductive material, for example, polypyrrole, carbonized ink, or metallized ink. In many embodiments, the conductive fabric is a polymeric fabric coated with a conductive polymeric material such as polypyrrole. In addition, the flexible heating element may be made from a matrix of electrically resistant wire or metal traces attached to a fibrous or film material layer.

FIG. 3A further illustrates subassembly 300 including two bus bars 315 coupled to heating element 310 for powering heating element 310; each bar 315 is shown extending between first and second ends 301, 302. With reference to FIG. 3B, according to some embodiments, bus bars 315 are coupled to heating element 310 by a stitched coupling 345, for example, formed with conductive thread such as silver-coated polyester or nylon thread (Marktek Inc., Chesterfield, Mo.). FIG. 3B illustrates subassembly 300 wherein insulating members 318, for example, fiberglass material strips having an optional PTFE coating and a thickness of approximately 0.003 inch, extend between bus bars 315 and heating element 310 at each stitched coupling 345, so that electrical contact points between bars 315 and heating element 310 are solely defined by the conductive thread of stitched couplings 345. Alternatively, the electrical insulation material layer could be made of polymeric film, a polymeric film reinforced with a fibrous material, a cellulose material, a glass fibrous material, rubber sheeting, polymeric or rubber coated fabric or woven materials or any other suitable electrically insulating material. Each of the conductive thread stitches of coupling 345 maintains a stable and constant contact with bus bar 315 on one side and heating element 310 on the other side of insulating member 318. Specifically, the stitches produce a stable contact in the face of any degree of flexion, so that the potential problem of intermittent contact between bus bar 315 and heating element 310 can be avoided. The stitches are the only electrical connection between bus bar 315 and heating element 310, but, since the conductive thread has a much lower electrical resistance than the conductive fabric of heating element 310, the thread does not heat under normal conditions. In addition to heating blanket applications described herein, such a design for providing for a uniform and stable conductive interface between a bus bar and a conductive fabric heater material can be used to improve the conductive interface between a bus bar or electrode and a conductive fabric in non-flexible heaters, in electronic shielding, in radar shielding and other applications of conductive fabrics.

Preferably, coupling 345 includes two or more rows of stitches for added security and stability. However, due to the flexible nature of blanket subassembly 300, the thread of stitched couplings 345 may undergo stresses that, over time and with multiple uses of a blanket containing subassembly 300, could lead to one or more fractures along the length of stitched coupling 345. Such a fracture, in other designs, could also result in intermittent contact points, between bus bar 315 and heating element 310, that could lead to a melt down of heating element 310 along bus bar. But, if such a fracture were to occur in the embodiment of FIG. 3B, insulating member 318 may prevent a meltdown of heating element 310, so that only the conductive thread of stitched coupling 345 melts down along bus bar 315.

Alternative threads or yarns employed by embodiments of the present invention may be made of other polymeric or natural fibers coated with other electrically conductive materials; in addition, nickel, gold, platinum and various conductive polymers can be used to make conductive threads. Metal threads such as stainless steel, copper or nickel could also be used for this application. According to an exemplary embodiment, bars 315 are comprised of flattened tubes of braided wires, such as are known to those skilled in the art, for example, a flat braided silver coated copper wire, and may thus accommodate the thread extending therethrough, passing through openings between the braided wires thereof. In addition such bars are flexible to enhance the flexibility of blanket subassembly 300. According to alternate embodiments, bus bars 315 can be a conductive foil or wire, flattened braided wires not formed in tubes, an embroidery of conductive thread, or a printing of conductive ink. Preferably, bus bars 315 are each a flat braided silver-coated copper wire material, since a silver coating has shown superior durability with repeated flexion, as compared to tin-coated wire, for example, and may be less susceptible to oxidative interaction with a polypyrrole coating of heating element 310 according to an embodiment described below. Additionally, an oxidative potential, related to dissimilar metals in contact with one another is reduced if a silver-coated thread is used for stitched coupling 345 of a silver-coated bus bar 315.

The shape of a surface area of heating element 310 is suited for use as a heating assembly 150 of a lower body heating blanket, for example, blanket 100 shown in FIG. 1, that would cover the lower torso of a patient undergoing lower body surgery.

According to an exemplary embodiment, a conductive fabric comprising heating element 310 comprises a non-woven polyester having a basis weight of approximately 170 g/m² and being 100% coated with polypyrrole (available from Eeonyx Inc., Pinole, Calif.); the coated fabric has an average resistance, for example, determined with a four point probe measurement, of approximately 15 ohms per square inch, which is suitable to produce the preferred watt density of 0.2 to 0.4 watts/sq. in. for surface areas of heating element 310 having a width, between bus bars 315, in the neighborhood of about 18 to 24 inches, when powered at about 48 volts. In some embodiments, the basis weight of the non-woven polyester may be chosen in the range of approximately 80-180 g/m². However, other basis weights may be engineered to operate adequately are therefore within the scope of embodiments of the invention.

According to an exemplary embodiment for an adult lower body heating blanket, a distance between a first end 301 of heating element 310 and a second end 302 of heating element 310 is between about 24 to 36 inches, while a distance between the bus bars 315 is about 18 to 24 inches. Such a width is suitable for a lower body heating blanket, some embodiments of which will be described below. A resistance of such a conductive fabric may be tailored for different widths between bus bars (wider requiring a lower resistance and narrower requiring a higher resistance) by increasing or decreasing a surface area of the fabric that can receive the conductive coating, for example by increasing or decreasing the basis weight of the nonwoven. Resistance over the surface area of the conductive fabrics is generally uniform in many embodiments of the present invention. However, the resistance over different portions of the surface area of conductive fabrics such as these may vary, for example, due to variation in a thickness of a conductive coating, variation within the conductive coating itself, variation in effective surface area of the substrate which is available to receive the conductive coating, or variation in the density of the substrate itself. Local surface resistance across a heating element, for example heating element 310, is directly related to heat generation according to the following relationship:

Q(Joules)=I²(Amps)×R(Ohms)

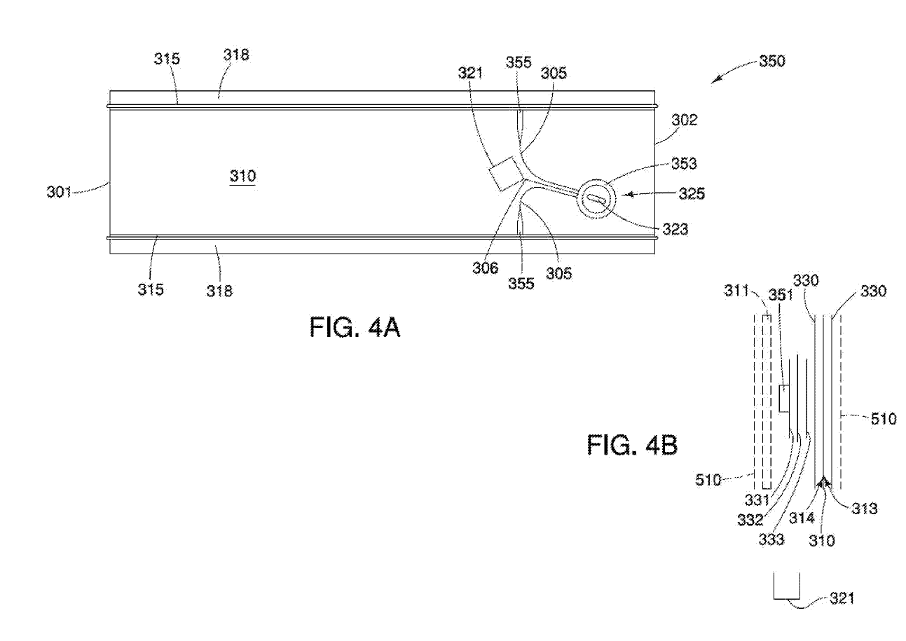

Variability in resistance thus translates into variability in heat generation, which manifests as a variation in temperature. According to preferred embodiments of the present invention, which are employed to warm patients undergoing surgery, precise temperature control is desirable. Means for determining heating element temperatures, which average out temperature variability caused by resistance variability across a surface of the heating element, are described below in conjunction with FIG. 4A.

A flexibility of blanket subassembly 300, provided primarily by flexible heating element 310, and optionally enhanced by the incorporation of flexible bus bars, allows blanket subassembly 300 to conform to the contours of a body, for example, all or a portion of a patient undergoing surgery, rather than simply bridging across high spots of the body; such conformance may optimize a conductive heat transfer from heating element 310 to a surface of the body.

The uniform watt-density output across the surface areas of preferred embodiments of heating element 310 translates into generally uniform heating of the surface areas, but not necessarily a uniform temperature. At locations of heating element 310 which are in conductive contact with a body acting as a heat sink, for example the heat is efficiently drawn away from heating element 310 and into the body, for example by blood flow, while at those locations where heating element 310 does not come into conductive contact with the body, an insulating air gap exists between the body and those portions, so that the heat is not drawn off those portions as easily. Therefore, those portions of heating element 310 not in conductive contact with the body will gain in temperature, since heat is not transferred as efficiently from these portions as from those in conductive contact with the body. The ‘non-contacting’ portions will reach a higher equilibrium temperature than that of the ‘contacting’ portions, when the radiant and convective heat loss equal the constant heat production through heating element 310. Since the heat generation is generally uniform, the heat loss in a steady state will be generally uniform, and therefore the flux to the patient will also be generally uniform. However, at the non-contacting locations, the temperature is higher to achieve the same flux as the contacting portions. Some of the extra heat at the non-contacting portions is therefore dissipated out the back of the pad instead of into the patient. Although radiant and convective heat transfer are more efficient at higher heater temperatures, the laws of thermodynamics dictate that as long as there is a uniform watt-density of heat production, even at the higher temperature, the radiant and convective heat transfer from a blanket of this construction will result in a generally uniform heat flux from the blanket. Therefore, by controlling the ‘contacting’ portions to a safe temperature, for example, via a temperature sensor assembly 321 coupled to heating element 310 in a location where heating element 310 will be in conductive contact with the body as shown in FIG. 4A, the ‘non-contacting’ portions, will also be operating at a safe temperature because of the less efficient radiant and convective heat transfer. According to preferred embodiments, heating element 310 comprises a conductive fabric having a relatively small thermal mass so that when a portion of the heating element that is operating at the higher temperature is touched, suddenly converting a ‘non-contacting’ portion into a ‘contacting’ portion, that portion will cool almost instantly to the lower operating temperature. According to the embodiment illustrated in FIG. 4A, temperature sensor assembly 321 is coupled to heating element 310 at a location where heating element 310, when incorporated in a lower body heating blanket, for example, blanket 100, would come into conductive contact with the lower body of a patient such as the patient's thigh, in order to maintain a safe temperature distribution across heating element 310.

With reference to FIG. 4A, in conjunction with FIG. 1, it may be appreciated that temperature sensor assembly 321 is located on assembly 350 so that, when blanket 100 including assembly 350 is draped over the lower body of the patient, the area of heating element 310 surrounding sensor assembly 321 will be in conductive contact with the lower body of the patient such as the patient's leg (the upper thigh in some embodiments) in order to maintain a safe temperature distribution across heating element 310.

According to embodiments of the present invention, sections of heating element 310 may be differentiated according to whether or not portions of heating element 310 are in conductive contact with a body, for example, a patient undergoing surgery. In the case of conductive heating, gentle external pressure may be applied to a heating blanket including heating element 310, which pressure forces heating element 310 into better conductive contact with the patient to improve heat transfer. However, if excessive pressure is applied the blood flow to that skin may be reduced at the same time that the heat transfer is improved and this combination of heat and pressure to the skin can be dangerous. It is well known that patients with poor perfusion should not have prolonged contact with conductive heat in excess of approximately 42° C. 42° C. has been shown in several studies to be the highest skin temperature, which cannot cause thermal damage to normally perfused skin, even with prolonged exposure. (Stoll & Greene, Relationship between pain and tissue damage due to thermal radiation. J. Applied Physiology 14(3):373-382. 1959. and Moritz and Henriques, Studies of thermal injury: The relative importance of time and surface temperature in the causation of cutaneous burns. Am. J. Pathology 23:695-720, 1947). Thus, according to certain embodiments of the present invention, the portion of heating element 310 that is in conductive contact with the patient is controlled to approximately 43° C. in order to achieve a temperature of about 41-42° C. on a surface a heating blanket cover that surrounds heating element 310, for example, a cover or shell 50 which was described above in conjunction with FIG. 1.

FIG. 4A is a top plan view of a heating element assembly 350, according to some embodiments of the present invention, which may be used as assembly 150 in blanket 100, which is shown, for example, in FIGS. 1 and 2A-2D. FIGS. 4A and 4B illustrate a temperature sensor assembly 321 assembled on side 314 of heating element and heating element 310 overlaid on both sides 313, 314 with an electrically insulating layer 330, preferably formed of a flexible non-woven, or non-woven fibrous material, for example, 1.5 OSY (ounces per square yard) nylon, which is preferably laminated to sides 313, 314 with a hotmelt laminating adhesive. In some embodiments, the adhesive is applied over the entire interfaces between insulating layer 330 and heating element 310. Other examples of suitable materials for insulating layer 330 include, without limitation, polymeric foam, a woven fabric, such as cotton or fiberglass, and a relatively thin plastic film, cotton, and a non-flammable material, such as fiberglass or treated cotton. According to preferred embodiments, overlaid insulating layers 330, without compromising the flexibility of heating assembly 350, prevent electrical shorting of one portion of heating element 310 with another portion of heating element 310 if heating element 310 is folded over onto itself. Heating element assembly 350 may be enclosed within a relatively durable and waterproof shell, for example shell 50 shown with dashed lines in FIG. 4B, and will be powered by a relatively low voltage (approximately 48V). Insulating layers 330 may even be porous in nature to further maintain the desired flexibility of assembly 350.

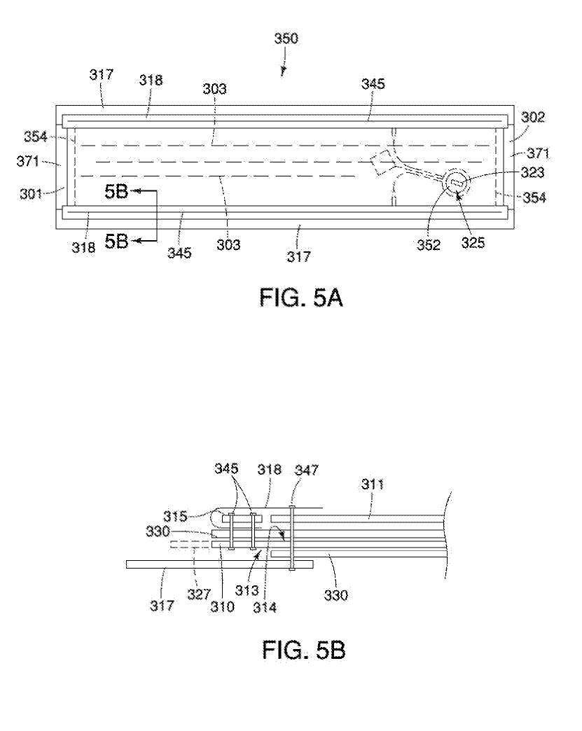

FIG. 4A further illustrates junctions 355 coupling leads 305 to each bus bar 315, and another lead 306 coupled to and extending from temperature sensor assembly 321; each of leads 305, 306 extend over insulating layer 330 and into an electrical connector housing 325 containing a connector plug 323, which will be described in greater detail below, in conjunction with FIG. 5A. Returning now to FIG. 4B, temperature sensor assembly 321 will be described in greater detail. FIG. 4B illustrates sensor assembly 321 including a substrate 331, for example, of polyimide (Kapton), on which a temperature sensor 351, for example, a surface mount chip thermistor (such as a Panasonic ERT-J1VG103FA: 10K, 1% chip thermistor), is mounted; a heat spreader 332, for example, a copper or aluminum foil, is mounted to an opposite side of substrate 331, for example, being bonded with a pressure sensitive adhesive; substrate 331 is relatively thin, for example about 0.0005 inch thick, so that heat transfer between heat spreader 332 and sensor is not significantly impeded.

Sensor 351, according to embodiments of the present invention, is positioned such that, when a heating blanket including heating element 310 is placed over a body, the regions surrounding sensor 351 will be in conductive contact with the body. As previously described, it is desirable that a temperature of approximately 43° C. be maintained over a surface of heating element 310 which is in conductive contact with a body of a patient undergoing surgery. An additional alternate embodiment is contemplated in which an array of temperature sensors are positioned over the surface of heating element 310, being spaced apart so as to collect temperature readings which may be averaged to account for resistance variance.

FIG. 5A is a top plan view of heating element assembly 350, which may be incorporated into blanket 100; and FIG. 5B is a cross-section view through section line 5B-5B of FIG. 5A. FIGS. 5A-B illustrate heating element assembly 350 including heating element 310 overlaid with electrical insulation layering 330 on both sides 313, 314 and thermal insulation layer 311 extending over the top side 314 thereof (dashed lines show leads and sensor assembly beneath layer 311). Blanket 100 may include a layer of thermal insulation 311 extending over a top side (corresponding to side 314 of heating element 310 as shown in FIG. 3B) of assembly 150. The layer of thermal insulation may or may not be bonded to a surface of assembly 150. It may serve to prevent heat loss away from a body disposed on the opposite side of blanket 100, particularly if a heat sink comes into contact with the top side of blanket 100. The insulation layer may extend over an entire surface of side 314 (FIG. 3B) of heating element 310 (top surface) and over sensor assembly 321 (FIGS. 4A, 4B) and may be secured to heating element assembly 150, as will be described in greater detail below. In some embodiments, layer 311 comprises any, or a combination of the following: a non-woven material (e.g., a CDS200 Thinsulate by 3M), other high loft fibrous polymeric non-woven materials, non-woven cellulose material, and air, for example, held within a polymeric film bubble. In some embodiments, the insulating layer comprises a polymer foam, for example, a 2 pound density 50 ILD urethane foam, which has a thickness between approximately ⅛^{th} inch and approximately ¾^{th} inch.

According to the illustrated embodiment, layer 311 is inserted beneath a portion of each insulating member 318, each which has been folded over the respective bus bar 315, for example as illustrated by arrow B in FIG. 3B, and then held in place by a respective row of non-conductive stitching 347 that extends through insulating member 318, layer 311 and heating element 310. Although not shown, it should be appreciated that layer 311 may further extend over bus bars 315. Although insulating layer 330 is shown extending beneath layer 311 on side 314 of heating element, according to alternate embodiments, layer 311 independently performs as a thermal and electrical insulation so that insulating layer 330 is not required on side 314 of heating element 310. FIG. 5A further illustrates, with longitudinally extending dashed lines, a plurality of optional slits 303 in layer 311, which may extend partially or completely through layer 311, in order to increase the flexibility of assembly 350. Such slits are desirable if a thickness of layer 311 is such that it prevents blanket 100 from draping effectively about a patient; the optional slits are preferably formed, for example, extending only partially through layer 311 starting from an upper surface thereof, to allow bending of blanket 100 about a patient and to prevent bending of blanket 100 in the opposition direction.

Returning now to FIG. 4A, to be referenced in conjunction with FIG. 5A, connector housing 325 and connector plug 323 will be described in greater detail. According to certain embodiments, housing 325 is an injection molded thermoplastic, for example, PVC, and may be coupled to assembly 350 by being stitched into place, over insulating layer 330. FIG. 4A shows housing 325 including a flange 353 through which such stitching can extend. Connector plug 323 protrudes from shell 50 of blanket 100 so that an extension cable may couple bus bars to a power source, and temperature sensor assembly 321 to a temperature controller, both of which may be incorporated into a console. In certain embodiments, the power source supplies a pulse-width-modulated voltage to bus bars 315. The controller may function to interrupt such power supply (e.g., in an over-temperature condition) or to modify the duty cycle to control the heating element temperature. In some embodiments, a surface of flange of housing 325 (FIG. 5A) protrudes through a hole formed in thermal insulating layer 311 so that a seal may be formed, for example, by adhesive bonding and/or heat sealing, between an inner surface of shell 50 and surface 352. According to one embodiment, wherein housing 325 is injection molded PVC and the inner surface of shell 50 is coated with polyurethane, housing 325 is sealed to shell portion 50 via a solvent bond. It may be appreciated that the location of the connector plug 323 is suitable to keep the corresponding connector cord well away from the surgical field.

FIGS. 5A-B further illustrate a pair of securing strips 317, each extending laterally from and alongside respective lateral portions of heating element 310, parallel to bus bars 315, and each coupled to side 313 of heating element 310 by the respective row of non-conductive stitching 347. Another pair of securing strips 371 is shown in FIG. 5A, each strip 371 extending longitudinally from and alongside respective ends 301, 302 of heating element 310 and being coupled thereto by a respective row of non-conductive stitching 354. Strips 371 may extend over layer 311 or beneath heating element 310. Strips 317 preferably extend over conductive stitching of stitched coupling 345 on side 313 of heating element 310, as shown, to provide a layer of insulation that can prevent shorting between portions of side 313 of heating element 310 if heating element 310 were to fold over on itself along rows of conductive stitching of stitched coupling 345 that couple bus bars 315 to heating element 310; however, strips 317 may alternately extend over insulating member 318 on the opposite side of heating element 310. According to the illustrated embodiment, securing strips 317 and 371 are made of a polymer material, for example, PVC. They may be heat sealed between the sheets of shell 50 in corresponding areas of the heat seal zone in order to secure heating element assembly 350 within a corresponding gap between the two sheets of shell 50. According to an alternate embodiment, for example, shown by dashed lines in FIGS. 3A and 5B, heating element 310 extends laterally out from each bus bar 315 to a securing edge 327, which may include one or more slots or holes 307 extending therethrough so that inner surfaces of sheets of shell 50 can contact one another to be sealed together and thereby hold edges 327.

In the foregoing detailed description, the invention has been described with reference to specific embodiments. However, it may be appreciated that various modifications and changes can be made without departing from the scope of the invention as set forth. Although embodiments of the invention are described in the context of a hospital operating room, it is contemplated that some embodiments of the invention may be used in other environments. Those embodiments of the present invention, which are not intended for use in an operating environment and need not meet stringent FDA requirements for repeated used in an operating environment, need not include particular features described herein, for example, related to precise temperature control. Thus, some of the features of preferred embodiments described herein are not necessarily included in preferred embodiments of the invention which are intended for alternative uses.

1. A tuckable electric heating blanket, comprising:
a flexible sheet-like heating element;
a flexible shell covering the heating element;
one or more stays extending along right and left edges of the heating element, the stays being relatively stiff; and
unheated fold zones positioned between the heating element and the stays.

2. The blanket of claim 1, wherein two stays extend along each of the right and left edges of the heating element, the two stays being separated by a longitudinal gap, the longitudinal gaps on the right and left edges being longitudinally aligned to form a natural folding location that allows the blanket to be folded back onto itself.

3. The blanket of claim 1, wherein one stay extends along each of the right and left edges of the heating element, the one stay generally preventing the blanket from being folded over onto itself.

4. The blanket of claim 1, wherein the one or more stays are generally planar.

5. The blanket of claim 1, wherein the one or more stays extend along approximately the entire right and left edges of the heating element.

6. The blanket of claim 1, wherein the flexible shell includes top and bottom sheets extending over both upper and lower faces of the heating element assembly, the top and bottom sheets coupled together along a seal zone that extends around the perimeter of the heating element, about the outer edge of the blanket, and around the one or more stays to hold the stays in a generally fixed position relative to the blanket.

7. The blanket of claim 1, further comprising an unheated foot portion pocket located longitudinally of one of the longitudinal ends of the heating element, the foot portion pocket providing an unheated flexible portion of the blanket tuckable about the patient's feet to retain heat under the blanket.

8. An electric heating blanket shaped to cover the lower body of a patient, comprising:
an electric heating element assembly for covering the legs and hips of the patient, the heating element assembly having opposing right and left edges and opposing longitudinal ends;
a flexible shell including top and bottom sheets extending over both upper and lower faces of the heating element assembly, the top and bottom sheets coupled together around the perimeter of the heating element assembly and about the outer edge of the blanket, the flexible shell forming pockets about zones where the top and bottom sheets remain uncoupled together; and
an unheated foot portion pocket located longitudinally of one of the longitudinal ends of the heating element assembly, the foot portion pocket providing an unheated flexible portion of the blanket tuckable about the patient's feet to retain heat under the blanket.

9. The blanket of claim 8, further comprising one or more stays extending along right and left edges of the heating element assembly, the stays being relatively stiff.

10. The blanket of claim 9, further comprising unheated fold zones positioned between the heating element and the one or more stays.

11. The blanket of claim 9, wherein the one or more stays do not extend longitudinally past the right and left edges of the heating element.

12. The blanket of claim 9, wherein the one or more stays do not extend into the unheated foot portion.

13. A method of using a tuckable electric heating blanket, comprising:
placing a patient on a surface with the patient's body extended against the surface;
placing the electric heating blanket over the patient's body, the blanket including a flexible heating element and one or more longitudinal stays extending along right and left edges of the heating element, the blanket having unheated fold zones positioned between the heating element and the stays;
positioning the blanket with the heating element over at least a portion of the patient's body;
positioning the one or more longitudinal stays on the surface beside the at least a portion of the patient's body; and
tucking the blanket beneath the at least a portion of the patient's body by sliding the one or more stays over the surface and beneath the at least a portion of the patient's body until there is resistance to advancing the stay.

14. The method of claim 13, wherein tucking the blanket includes folding the blanket back upon itself along the fold zones.

15. The method of claim 14, wherein the folding brings portions of the upper face of the blanket along the fold zones into contact with each other.

16. The method of claim 13, wherein tucking the blanket includes pushing the blanket along the fold zones underneath the patient.

17. The method of claim 13, wherein stays are generally planar and the resistance to advancing the stay is generated from the generally planar stays resisting folding.
